(19) United States
(12) Patent Application Publication
(10) Pub. No.: US 2025/0249260 A1
(43) Pub. Date: Aug. 7, 2025

(54) METHOD AND APPARATUS FOR PROGRAMMING NEUROSTIMULATION WITH DYNAMIC STIMULATION PATTERNS

(71) Applicant: Boston Scientific Neuromodulation Corporation, Valencia, CA (US)

(72) Inventors: Satya Venkata Sandeep Avvaru, Houston, TX (US); Tianhe Zhang, Studio City, CA (US); Rosana Esteller, Santa Clarita, CA (US)

(21) Appl. No.: 19/033,131

(22) Filed: Jan. 21, 2025

Related U.S. Application Data
(60) Provisional application No. 63/549,910, filed on Feb. 5, 2024.

(51) Int. Cl. A61N 1/36; A61N 1/02; A61N 1/372

(52) U.S. Cl. CPC A61N 1/36185; A61N 1/025; A61N 1/36071; A61N 1/37247

(57) ABSTRACT

A system for delivering neurostimulation from a stimulation device to a targeted area on a patient may include a programming control circuit and a stimulation programming circuit. The programming control circuit may be configured to generate information for programming the stimulation device to control the delivery of the neurostimulation according to a stimulation program. The stimulation programming circuit may be configured to receive a stimulation field and a stimulation coverage and to determine the stimulation program by including at least one dynamic stimulation pattern configured to expand the stimulation coverage resulting from delivering the neurostimulation to the stimulation field. The stimulation coverage is a portion of the targeted area effectively stimulated by the neurostimulation delivered to the stimulation field. The dynamic stimulation pattern is defined by at least one time-varying stimulation waveform parameter of the stimulation waveform parameters.

### CLAIM OF PRIORITY

This application claims the benefit of U.S. Provisional Application No. 63/549,910, filed on Feb. 5, 2024, which is hereby incorporated by reference in its entirety.

### TECHNICAL FIELD

This document relates generally to neurostimulation and more particularly to a system and method for programming neurostimulation with dynamic stimulation patterns each being a pattern of neurostimulation pulses defined by stimulation parameters including one or more time-varying parameters.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

In one example, the neurostimulation energy is delivered to a patient in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Neurostimulation controlled using a pattern of neurostimulation pulses defined by constant stimulation parameters has been applied to effectively treat various disorders, while use of time-varying stimulation parameters can better resemble natural neural activities and hence have the potential of providing for better therapeutic effectiveness, when the stimulation parameters are properly programmed to utilize the beneficial effects of neurostimulation using one or more time-varying stimulation parameters.

### SUMMARY

An Example (e.g., “Example 1”) of a system for delivering neurostimulation from a stimulation device to a targeted area on a patient is provided. The system may include a programming control circuit and a stimulation programming circuit. The programming control circuit may be configured to generate information for programming the stimulation device to control the delivery of the neurostimulation according to a stimulation program specifying stimulation waveform parameters defining at least one stimulation waveform of the neurostimulation and stimulation field parameters defining at least one stimulation field to which the neurostimulation is to be delivered. The stimulation programming circuit may be configured to receive a stimulation field and a stimulation coverage and to determine the stimulation program by including at least one dynamic stimulation pattern configured to expand the stimulation coverage resulting from delivering the neurostimulation to the stimulation field. The stimulation field is defined by the stimulation field parameters. The stimulation coverage is a portion of the targeted area effectively stimulated by the neurostimulation delivered to the stimulation field. The dynamic stimulation pattern is defined by at least one time-varying stimulation waveform parameter of the stimulation waveform parameters.

In Example 2, the subject matter of Example 1 may optionally be configured such that the at least one time-varying stimulation parameter is modulated by a modulation function.

In Example 3, the subject matter of Example 2 may optionally be configured such that the at least one time-varying stimulation parameter is a pulse amplitude, a pulse width, or a pulse frequency.

In Example 4, the subject matter of any one or any combination of Examples 1 to 3 may optionally be configured such that the stimulation programming circuit is configured to determine the stimulation program by including a cycling sequence of different stimulation patterns including the at least one dynamic stimulation pattern.

In Example 5, the subject matter of Example 4 may optionally be configured such that the cycling sequence of different stimulation patterns includes stimulation patterns each scheduled to be applied for an on-period during which the neurostimulation is delivered according to each stimulation pattern and separated from an adjacent stimulation pattern of the different stimulation patterns by an off-period during which no neurostimulation is delivered.

In Example 6, the subject matter of any one or any combination of Examples 1 to 5 may optionally be configured such that the stimulation programming circuit includes a target searching module configured to determine the stimulation field and to determine a paresthesia coverage as the stimulation coverage, the targeted area is an area of pain, and the paresthesia coverage is a portion of the area of pain where paresthesia is caused by the neurostimulation delivered to the stimulation field.

In Example 7, the subject matter of Example 6 may optionally be configured to include a user interface including a presentation device, a user input device, and an interface control circuit coupled to the presentation device and the user input device, the interface device including the stimulation programming circuit.

In Example 8, the subject matter of Example 7 may optionally be configured such that the target searching module is configured to perform a target searching including: presenting, using the presentation device, a representation of an anatomical region of the patient including the area of pain and a representation of electrodes each selectable for delivering the neurostimulation to the patient; receiving, using the user input device, a central point of stimulation (CPS) positioned on the anatomical region; determining the stimulation field based on the CPS; causing the stimulation device to deliver the neurostimulation to the stimulation field according to a tonic stimulation pattern; and determining the stimulation coverage resulting from the neurostimulation delivered to the stimulation field determined based on the CPS.

In Example 9, the subject matter of Example 8 may optionally be configured such that the target searching module is configured to allow for repeating the performance of the target searching for another CPS until the stimulation coverage exceeds a specified coverage threshold.

In Example 10, the subject matter of Example 8 may optionally be configured such that the target searching module is configured to repeat the performance of the target searching for multiple CPSs and determine the stimulation field as the stimulation field resulting in a maximum stimulation coverage identified from the stimulation coverages determined from the repeated performance of the target searching.

In Example 11, the subject matter of any one or any combination of Examples 1 to 10 may optionally be configured such that the stimulation programming circuit includes a coverage-based patterning module configured to receive the stimulation coverage and to determine the stimulation program using the received stimulation coverage and a coverage-based decision tree mapping ranges of the stimulation coverage to pre-configured stimulation programs each including one or more stimulation patterns.

In Example 12, the subject matter of Example 11 may optionally be configured such that the coverage-based patterning module is configured to be activated in response to the stimulation coverage being below a specified satisfactory stimulation coverage.

In Example 13, the subject matter of Example 12 may optionally be configured such that the pre-configured stimulation programs each including a cycling sequence of different stimulation patterns.

In Example 14, the subject matter of any one or any combination of Examples 1 to 12 may optionally be configured such that the stimulation programming circuit comprises an indication-based patterning module configured to receive an indication of the patient selected from a plurality of indications and to determine the stimulation program using the received indication and an indication-based decision tree mapping the indications to pre-configured stimulation programs each including one or more stimulation patterns.

In Example 15, the subject matter of Example 14 may optionally be configured such that the pre-configured stimulation programs each including a cycling sequence of different stimulation patterns.

An example (e.g., “Example 16”) of a method for delivering neurostimulation from a stimulation device to a targeted area on a patient is also provided. The method may include: receiving a stimulation field and a stimulation coverage, the stimulation coverage being a portion of the targeted area effectively stimulated by the neurostimulation delivered to the stimulation field; determining, using a processor of a programming device, a stimulation program specifying stimulation waveform parameters defining at least one stimulation waveform of the neurostimulation and stimulation field parameters defining the stimulation field, the stimulation program including at least one dynamic stimulation pattern configured to expand the stimulation coverage resulting from delivering the neurostimulation to the stimulation field, the dynamic stimulation pattern defined by at least one time-varying stimulation waveform parameter of the stimulation waveform parameters; and programming the stimulation device, using the programming device, to control the delivery of the neurostimulation according to the determined stimulation program.

In Example 17, the subject matter of Example 16 may optionally further include modulating a stimulation parameter using a modulating function to produce the at least one time-varying stimulation parameter.

In Example 18, the subject matter of any one or any combination of Examples 16 and 17 may optionally further include selecting a pre-configured cycling sequence of different stimulation patterns to be the stimulation program. The stimulation patterns are each scheduled to be applied for an on-period during which the neurostimulation is to be delivered according to each stimulation pattern and separated from an adjacent stimulation pattern of the different stimulation patterns by an off-period during which no neurostimulation is delivered.

In Example 19, the subject matter of any one or any combination of Examples 16 to 18 may optionally further include performing a target searching to determine the stimulation field and the stimulation coverage. The target searching include: presenting an anatomical region of the patient including the targeted area and a representation of electrodes each selectable for delivering the neurostimulation to the patient; receiving a central point of stimulation (CPS) positioned on the anatomical region; determining the stimulation field based on the CPS; causing the stimulation device to deliver the neurostimulation to the stimulation field according to a tonic stimulation pattern; and determining a stimulation coverage resulting from the neurostimulation delivered to the stimulation field determined based on the CPS.

In Example 20, the targeted area as found in Example 19 is an area of pain, and the subject matter of determining the stimulation coverage as found in Example 19 may optionally include determining a paresthesia coverage being a portion of the area of pain where paresthesia is caused by the neurostimulation delivered to the stimulation field.

In Example 21, the subject matter of any one or any combination of Examples 19 and 20 may optionally further include repeating the performance of the target searching for another CPS until the stimulation coverage exceeds a specified coverage threshold.

In Example 22, the subject matter of determining the stimulation program as found in any one or any combination of Examples 16 to 21 may optionally include determining the stimulation program using the received stimulation coverage and a coverage-based decision tree mapping ranges of the stimulation coverage to pre-configured stimulation programs each including one or more stimulation patterns.

In Example 23, the subject matter of Example 22 may optionally further include determining the stimulation program using the received stimulation coverage and a coverage-based decision tree in response to the stimulation coverage being below a specified minimum coverage.

In Example 24, the subject matter of determining the stimulation program as found in any one or any combination of Examples 16 to 23 may optionally include receiving an indication of the patient selected from a plurality of indications and determining the stimulation program using the received indication and an indication-based decision tree mapping the indications to pre-configured stimulation programs each including one or more stimulation patterns.

An example (e.g., “Example 25”) of a non-transitory computer-readable storage medium including instructions is also provided. The instructions, which when executed by a system, cause the system to perform a method for delivering neurostimulation from a stimulation device to a targeted area on a patient. The method comprising: receiving a stimulation field and a stimulation coverage resulting from delivering the neurostimulation to the stimulation field, the stimulation coverage being a portion of the targeted area effectively stimulated by the neurostimulation delivered to the stimulation field; determining, using a processor of a programming device, a stimulation program specifying stimulation waveform parameters defining at least one stimulation waveform of the neurostimulation and stimulation field parameters defining the stimulation field, the stimulation program including at least one dynamic stimulation pattern configured to expand the stimulation coverage resulting from delivering the neurostimulation to the stimulation field, the dynamic stimulation pattern defined by at least one time-varying stimulation waveform parameter of the stimulation waveform parameters; and programming the stimulation device, using the programming device, to control the delivery of the neurostimulation according to the determined stimulation program.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 illustrates an embodiment of a neurostimulation system.

FIG. 2 illustrates an embodiment of a stimulation device and a lead system, such as may be implemented in the neurostimulation system of FIG. 1.

FIG. 3 illustrates an embodiment of a programming device, such as may be implemented in the neurostimulation system of FIG. 1.

FIG. 4 illustrates an embodiment of an implantable pulse generator (IPG) and an implantable lead system, such as an example implementation of the stimulation device and lead system of FIG. 2.

FIG. 5 illustrates an embodiment of an IPG and an implantable lead system, such as the IPG and lead system of FIG. 4, arranged to provide neurostimulation to a patient.

FIG. 6 illustrates an embodiment of portions of a neurostimulation system.

FIG. 7 illustrates an embodiment of an implantable stimulator and one or more leads of an implantable neurostimulation system, such as the implantable neurostimulation system of FIG. 6.

FIG. 8 illustrates an embodiment of an external programming device of an implantable neurostimulation system, such as the implantable neurostimulation system of FIG. 6.

FIG. 9 illustrates an embodiment of a system for programming neurostimulation with one or more dynamic stimulation patterns.

FIG. 10 illustrates an embodiment of stimulation programming circuit of a neurostimulation system, such as the system of FIG. 9.

FIGS. 11A-D illustrate examples of various stimulation patterns, with FIG. 11A illustrating an example of a tonic stimulation pattern, FIG. 11B illustrating an example of an amplitude-modulated stimulation pattern, FIG. 11C illustrating an example of a frequency-modulated stimulation pattern, and FIG. 11D illustrating an example of a pulse width-modulated stimulation pattern.

FIGS. 12A-D illustrate examples of various dynamic stimulation patterns each with a modulated stimulation parameter, with FIG. 12A illustrating an example of a pulse amplitude modulated by a sinusoidal signal, FIG. 12B illustrating another example of a pulse amplitude modulated by a sinusoidal signal, FIG. 12C illustrating an example of a pulse rate modulated by a Poisson distribution signal, and FIG. 12D illustrating another example of a pulse rate modulated by a Poisson distribution signal.

FIGS. 13A-C illustrate an embodiment of a paresthesia-guided target searching process for pain management using spinal cord stimulation (SCS), with FIG. 13A illustrating an example of moving central point of stimulation (CPS) for an acceptable stimulation coverage, FIG. 13B illustrating an example in which a satisfactory stimulation coverage is achieved using a tonic stimulation pattern, and FIG. 13C illustrating an example in which a satisfactory stimulation coverage is not achieved using a tonic stimulation pattern.

FIG. 14 illustrates an embodiment of a method for determining a stimulation program including one or more dynamic stimulation patterns based on stimulation coverage achieved by neurostimulation.

FIG. 15 illustrates an embodiment of a method for determining a stimulation program including one or more dynamic stimulation patterns based on a patient's indication for neurostimulation.

FIG. 16 illustrates an embodiment of a method for determining a stimulation program using the methods illustrated in FIGS. 13-15.

FIG. 17 illustrates an embodiment of a method for presenting a patient's pain regions for determining the stimulation program.

FIG. 18 illustrates an embodiment of a method for expanding a stimulation coverage achieved by delivering neurostimulation to a stimulation field using one or more dynamic stimulation patterns.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses, among other things, a neurostimulation system that can deliver neurostimulation to a patient according to one or more dynamic stimulation patterns and programing the one or more dynamic stimulation patterns for expanding tissue areas that can be effectively stimulated by the neurostimulation. In various embodiments, the delivery of the neurostimulation includes delivering neurostimulation pulses. In various embodiments, the neuromodulation system can include an implantable device configured to deliver neurostimulation (also referred to as neuromodulation) therapies, such as spinal cord stimulation (SCS), deep brain stimulation (DBS), peripheral nerve stimulation (PNS), and vagus nerve stimulation (VNS), and one or more external devices configured to program or adjust the implantable device for its operations and monitor the performance of the implantable device. In this document, unless noted otherwise, a “patient” includes a person receiving treatment delivered from, and/or monitored using, a neurostimulation system according to the present subject matter. A “user” includes a physician, other caregiver who examines and/or treats the patient using the neurostimulation system, or other person who participates in the examination and/or treatment of the patient using the neurostimulation system (e.g., a technically trained representative, a field clinical engineer, a clinical researcher, or a field specialist from the manufacturer of the neurostimulation system).

Delivery of neurostimulation pulses controlled using stimulation parameters programmed to constant (not time-varying) values has been effective in treating various conditions, such as in treating pain using SCS. Dynamic stimulation patterns defined by time-varying stimulation parameters can be applied to produce more biomimetic effects on the nervous system and/or to improve therapeutic effects of SCS. However, a wide range of stimulation parameters defining a dynamic stimulation pattern requires substantially more complicated programming when compared to a stimulation pattern defined by constant stimulation parameters. Such a programming task can be cumbersome unless it can be substantially automated. What is desired is a “set and forget” automation, with which a neurostimulation program is created and programmed into an implantable neurostimulator in a patient and then adjusted automatically as needed, without the need of frequent adjustment and/or reprogramming using a source external to the neurostimulator. On the other hand, use of dynamic stimulation patterns can enable such automation, such as by producing desirable clinic effects that can reduce the need for frequent manual adjustments. Examples include the patient's responses to the neurostimulation that are more resilient to the patient's posture (thereby reducing the need for adjustment when the patient changes posture every time) and more tolerant to stimulation site (thereby reducing the need for precise targeting and need for frequent re-targeting due to lead migration and/or the patient's posture changes). Thus, there is a need for a feasible way of programming dynamic stimulation patterns for their clinical benefits.

The present subject matter relates to a system that can be used for programming neurostimulation for delivery according to dynamic stimulation patterns, such as for treating pain with SCS. The programming can include determining a temporal arrangement (e.g., alternating, cycling, and/or scheduling) of stimulation waveforms to compensate for incomplete spatial coverage of a stimulation target region by a single calibrated and/or titrated stimulation waveform. Various aspects of the programming according to the present subject matter can include, but are not limited to, determining a stimulation field approximately covering a target region using a stimulation waveform, determining temporal arrangement of stimulation waveforms and/or their associated stimulation intensities at the stimulation field, and customizing the temporal arrangement of stimulation waveforms based on the patient's needs.

Using dynamic stimulation pattern to cover stimulation target according to the present subject matter is advantageous over various programming methods that need precise target searching, which can be cumbersome and difficult. For example, the system and methods discussed herein can provide for pain relief using SCS without a precise searching for the best stimulation site and without the need for frequent adjustment of stimulation parameters (e.g., due to lead migration and/or patient posture changes) once initially set.

In this document, a “stimulation pattern” includes a sequence (or “train”) of neurostimulation pulses defined by stimulation parameters. A “tonic stimulation pattern” (also known as “tonic pulse sequence”, “tonic pulse train”, and the like) includes a pattern of neurostimulation pulses defined by stimulation parameters having constant values. A “dynamic stimulation pattern” (also known as “dynamic pulse sequence”, “dynamic pulse train”, and the like) includes a pattern of neurostimulation pulses defined by stimulation parameters including at least one stimulation parameter having a time-varying value. It is noted that in a tonic stimulation pattern, all the stimulation parameters have constant (i.e., not time-varying) values, while in a dynamic stimulation pattern, the stimulation parameters can each have a constant value or a time-varying value. In other words, at least one of the stimulation parameters of a dynamic stimulation pattern has a time-varying value, while the other stimulation parameters can each have a constant or time-varying value. One example of a dynamic stimulation pattern includes a “modulated stimulation pattern” (also known as “modulated pulse sequence”, “modulated pulse train, and the like), in which one or more of the stimulation parameters are each modulated by a modulation function. Examples of such modulated pulse sequence are discussed in U.S. patent application Ser. No. 17/530,236, “METHOD AND APPARATUS FOR GENERATING MODULATED NEUROSTIMULATION PULSE SEQUENCE”, filed on Nov. 18, 2021, published as US 2022/0184400 A1, assigned to Boston Scientific Neuromodulation Corporation, which is incorporated herein by reference in its entirety.

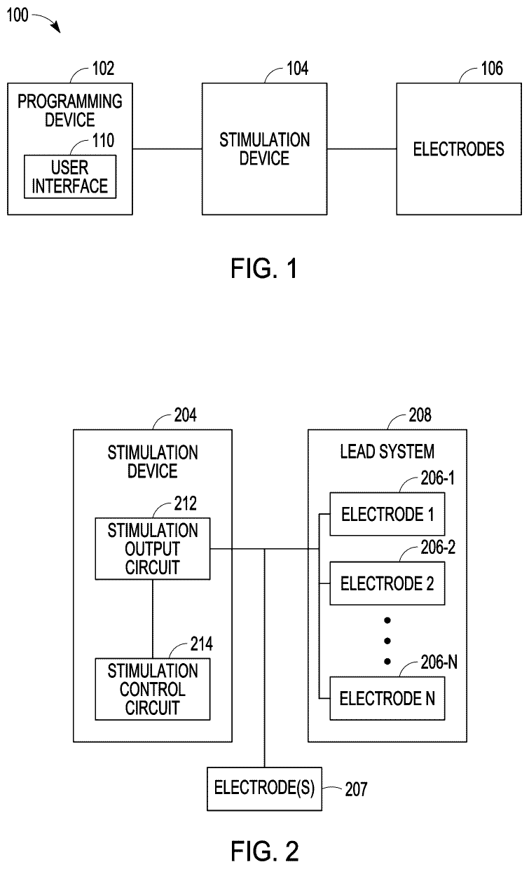

FIG. 1 illustrates an embodiment of a neurostimulation system 100. System 100 includes electrodes (also referred to as contacts) 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device via a wired or wireless link.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for spinal cord stimulation (SCS) applications. Such SCS configuration includes various features that may simplify the task of the user in programming stimulation device 104 for delivering SCS to the patient, such as the features discussed in this document.

FIG. 2 illustrates an embodiment of a stimulation device 204 and a lead system 208, such as may be implemented in neurostimulation system 100. Stimulation device 204 represents an example of stimulation device 104 and includes a stimulation output circuit 212 and a stimulation control circuit 214. Stimulation output circuit 212 produces and delivers neurostimulation pulses. Stimulation control circuit 214 controls the delivery of the neurostimulation pulses from stimulation output circuit 212 using the plurality of stimulation parameters, which specifies a pattern of the neurostimulation pulses. Lead system 208 includes one or more leads each configured to be electrically connected to stimulation device 204 and a plurality of electrodes 206 (also referred to as an electrode array in this document) distributed in the one or more leads. The plurality of electrodes 206 includes electrode 206-1, electrode 206-2, . . . electrode 206-N, each being a single electrically conductive contact providing for an electrical interface between stimulation output circuit 212 and tissue of the patient (and therefore also referred to as a contact), where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses or each of collections of pulse intended to be delivered using the same combination of electrodes. In various embodiments, one or more additional electrodes 207 (each of which may be referred to as a reference electrode) can be electrically connected to stimulation device 204, such as one or more electrodes each being a portion of or otherwise incorporated onto a housing of stimulation device 204. Monopolar stimulation uses a monopolar electrode configuration with one or more electrodes selected from electrodes 206 and at least one electrode from electrode(s) 207. Bipolar stimulation uses a bipolar electrode configuration with two electrodes selected from electrodes 206 and none of electrode(s) 207. Multipolar stimulation uses a multipolar electrode configuration with multiple (two or more) electrodes selected from electrodes 206 and none of electrode(s) 207.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes.

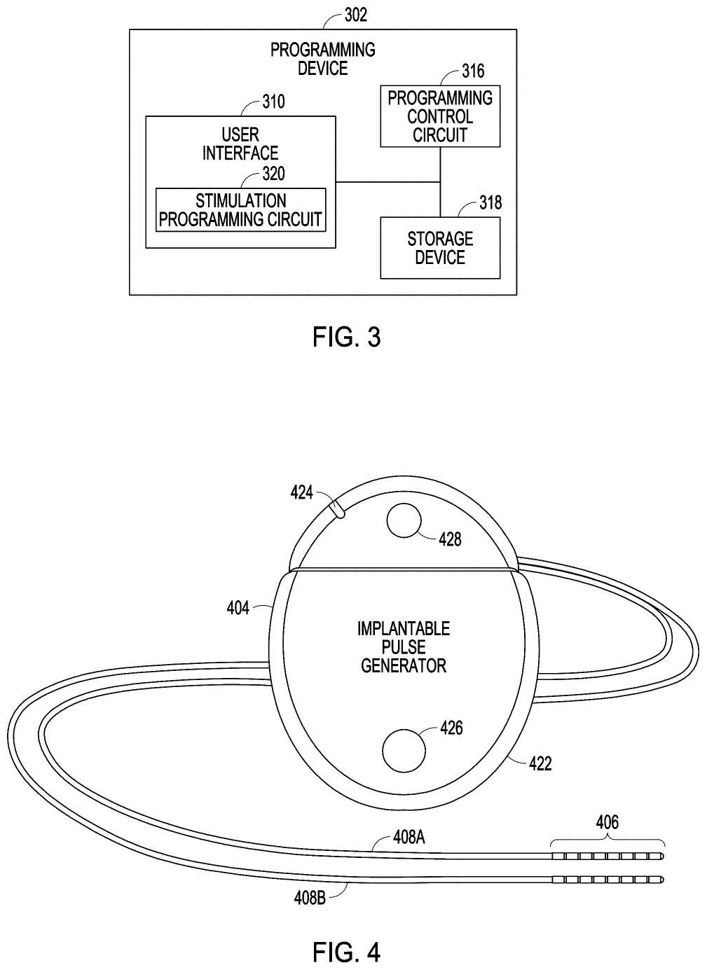

FIG. 3 illustrates an embodiment of a programming device 302, such as may be implemented in neurostimulation system 100. Programming device 302 represents an example of programming device 102 and includes a storage device 318, a programming control circuit 316, and a user interface 310. Programming control circuit 316 generates the plurality of stimulation parameters that controls the delivery of the neurostimulation pulses according to a specified neurostimulation program that can define, for example, stimulation waveform and electrode configuration. User interface 310 represents an example of user interface 110 and includes a stimulation programming circuit 320. Storage device 318 stores information used by programming control circuit 316 and stimulation programming circuit 320, such as information about a stimulation device that relates the neurostimulation program to the plurality of stimulation parameters. In various embodiments, stimulation programming circuit 320 can be configured to support one or more functions allowing for programming of stimulation devices, such as stimulation device 104 including its various embodiments as discussed in this document, to control delivery of neurostimulation according to the present subject matter (e.g., delivering neurostimulation pulses according to one or more dynamic stimulation patterns determined based on targeted neural response that can be sensed).

In various embodiments, user interface 310 can allow for definition of a pattern of neurostimulation pulses for delivery during a neurostimulation therapy session by creating and/or adjusting one or more stimulation waveforms using a graphical method. The definition can also include definition of one or more stimulation fields each associated with one or more pulses in the pattern of neurostimulation pulses. As used in this document, a “neurostimulation program” or “stimulation program” can include the pattern of neurostimulation pulses defined using the one or more stimulation waveforms and the one or more stimulation fields, or at least various spatial, temporal, and/or informational aspects or parameters of the pattern of neurostimulation pulses including the one or more stimulation waveforms and the one or more stimulation fields. In various embodiments, user interface 310 includes a GUI that allows the user to define the pattern of neurostimulation pulses and perform other functions using graphical methods. In this document, “neurostimulation programming” can include the definition of the one or more stimulation waveforms, including the definition of one or more stimulation fields.

In various embodiments, circuits of neurostimulation system 100, including its various embodiments discussed in this document, may be implemented using a combination of hardware and software. For example, the circuit of user interface 110, stimulation control circuit 214, programming control circuit 316, and stimulation programming circuit 320, including their various embodiments discussed in this document, can be implemented using an application-specific circuit constructed to perform one or more particular functions and/or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 4 illustrates an embodiment of an implantable pulse generator (IPG) 404 and an implantable lead system 408. IPG 404 represents an example implementation of stimulation device 204. Lead system 408 represents an example implementation of lead system 208. As illustrated in FIG. 4, IPG 404 that can be coupled to implantable leads 408A and 408B at a proximal end of each lead. The distal end of each lead includes electrodes 406 for contacting a tissue site targeted for electrical neurostimulation. As illustrated in FIG. 4, leads 408A and 408B each include 8 electrodes 406 at the distal end. The number and arrangement of leads 408A and 408B and electrodes 406 as shown in FIG. 4 are only an example, and other numbers and arrangements are possible. In various embodiments, the electrodes are ring electrodes. In various embodiments applying DBS or SCS, the implantable leads and electrodes may be configured by shape and size to provide electrical neurostimulation energy to a neuronal target included in the patient's brain or configured to provide electrical neurostimulation energy to target nerve cells in the patient's spinal cord.

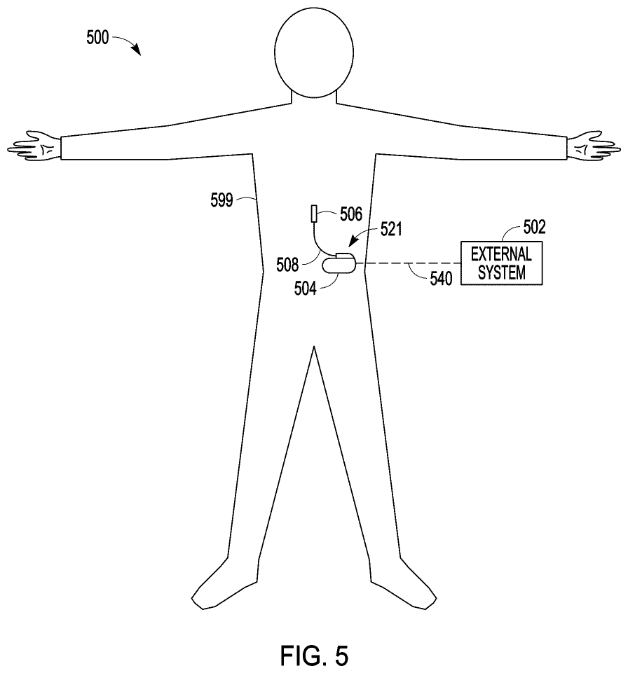

FIG. 5 illustrates an implantable neurostimulation system 500 and portions of an environment in which system 500 may be used. System 500 includes an implantable system 521, an external system 502, and a telemetry link 540 providing for wireless communication between implantable system 521 and external system 502. Implantable system 521 is illustrated in FIG. 5 as being implanted in the patient's body 599.

Implantable system 521 includes an implantable stimulator (also referred to as an implantable pulse generator, or IPG) 504, a lead system 508, and electrodes 506, which represent an example of stimulation device 204, lead system 208, and electrodes 206, respectively. External system 502 represents an example of programming device 302. In various embodiments, external system 502 includes one or more external (non-implantable) devices each allowing the user and/or the patient to communicate with implantable system 521. In some embodiments, external 502 includes a programming device intended for the user to initialize and adjust settings for implantable stimulator 504 and a remote control device intended for use by the patient. For example, the remote control device may allow the patient to turn implantable stimulator 504 on and off and/or adjust certain patient-programmable parameters of the plurality of stimulation parameters.

The sizes and shapes of the elements of implantable system 521 and their location in body 599 are illustrated by way of example and not by way of restriction. An implantable system is discussed as a specific application of the programming according to various embodiments of the present subject matter. In various embodiments, the present subject matter may be applied in programming any type of stimulation device that uses electrical pulses as stimuli, regarding less of stimulation targets in the patient's body and whether the stimulation device is implantable.

Returning to FIG. 4, the IPG 404 can include a hermetically-sealed IPG case 422 to house the electronic circuitry of IPG 404. IPG 404 can include an electrode 426 formed on IPG case 422. IPG 404 can include an IPG header 424 for coupling the proximal ends of leads 408A and 408B. IPG header 424 may optionally also include an electrode 428. Electrodes 426 and/or 428 represent embodiments of electrode(s) 207 and may each be referred to as a reference electrode.

Neurostimulation energy can be delivered in a monopolar (also referred to as unipolar) mode using electrode 426 or electrode 428 and one or more electrodes selected from electrodes 406. Neurostimulation energy can be delivered in a bipolar mode using a pair of electrodes of the same lead (lead 408A or lead 408B). Neurostimulation energy can be delivered in an extended bipolar mode using one or more electrodes of a lead (e.g., one or more electrodes of lead 408A) and one or more electrodes of a different lead (e.g., one or more electrodes of lead 408B).

The electronic circuitry of IPG 404 can include a control circuit that controls delivery of the neurostimulation energy. The control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

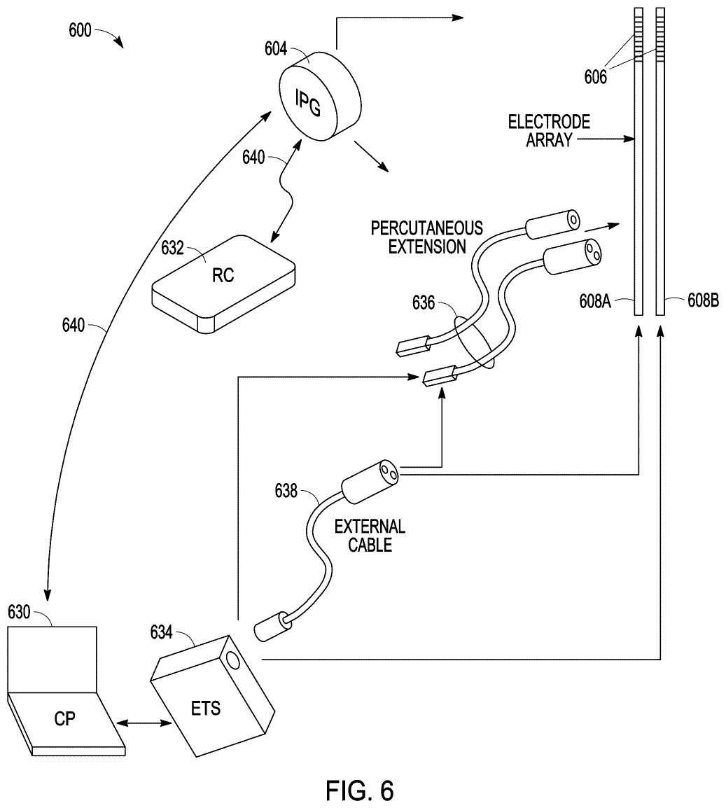

FIG. 6 illustrates an embodiment of portions of a neurostimulation system 600. System 600 includes an IPG 604, implantable neurostimulation leads 608A and 608B, an external remote controller (RC) 632, a clinician's programmer (CP) 630, and an external trial stimulator (ETS, also referred to as external trial modulator, ETM) 634. IPG 604 may be electrically coupled to leads 608A and 608B directly or through percutaneous extension leads 636. ETS 634 may be electrically connectable to leads 608A and 608B via one or both of percutaneous extension leads 636 and/or external cable 638. System 600 represents an example of system 100, with IPG 604 representing an embodiment of stimulation device 104, electrodes 606 of leads 608A and 608B representing electrodes 106, and CP 630, RC 632, and ETS 634 collectively representing programming device 102.

ETS 634 may be standalone or incorporated into CP 630. ETS 634 may have similar pulse generation circuitry as IPG 604 to deliver neurostimulation energy according to specified modulation parameters as discussed above. ETS 634 is an external device that is typically used as a preliminary stimulator after leads 408A and 408B have been implanted and used prior to stimulation with IPG 604 to test the patient's responsiveness to the stimulation that is to be provided by IPG 604. Because ETS 634 is external it may be more easily configurable than IPG 604.

CP 630 can configure the neurostimulation provided by ETS 634. If ETS 634 is not integrated into CP 630, CP 630 may communicate with ETS 634 using a wired connection (e.g., over a USB link) or by wireless telemetry using a wireless communications link 640. CP 630 also communicates with IPG 604 using a wireless communications link 640.

An example of wireless telemetry is based on inductive coupling between two closely-placed coils using the mutual inductance between these coils. This type of telemetry is referred to as inductive telemetry or near-field telemetry because the coils must typically be closely situated for obtaining inductively coupled communication. IPG 604 can include the first coil and a communication circuit. CP 630 can include or otherwise electrically connected to the second coil such as in the form of a wand that can be place near IPG 604. Another example of wireless telemetry includes a far-field telemetry link, also referred to as a radio frequency (RF) telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=λ/2π, where λ is the wavelength of the transmitted electromagnetic energy. In one example, a communication range of an RF telemetry link is at least six feet but can be as long as allowed by the particular communication technology. RF antennas can be included, for example, in the header of IPG 604 and in the housing of CP 630, eliminating the need for a wand or other means of inductive coupling. An example is such an RF telemetry link is a Bluetooth® wireless link.

CP 630 can be used to set modulation parameters for the neurostimulation after IPG 604 has been implanted. This allows the neurostimulation to be tuned if the requirements for the neurostimulation change after implantation. CP 630 can also upload information from IPG 604.

RC 632 also communicates with IPG 604 using a wireless link 640. RC 632 may be a communication device used by the user or given to the patient. RC 632 may have reduced programming capability compared to CP 630. This allows the user or patient to alter the neurostimulation therapy but does not allow the patient full control over the therapy. For example, the patient may be able to increase the amplitude of neurostimulation pulses or change the time that a preprogrammed stimulation pulse train is applied. RC 632 may be programmed by CP 630. CP 630 may communicate with the RC 632 using a wired or wireless communications link. In some embodiments, CP 630 can program RC 632 when remotely located from RC 632. In various embodiments, RC632 can be a dedicated device or a general-purpose device configured to perform the functions of RC 632, such as a smartphone, a tablet computer, or other smart/mobile device.

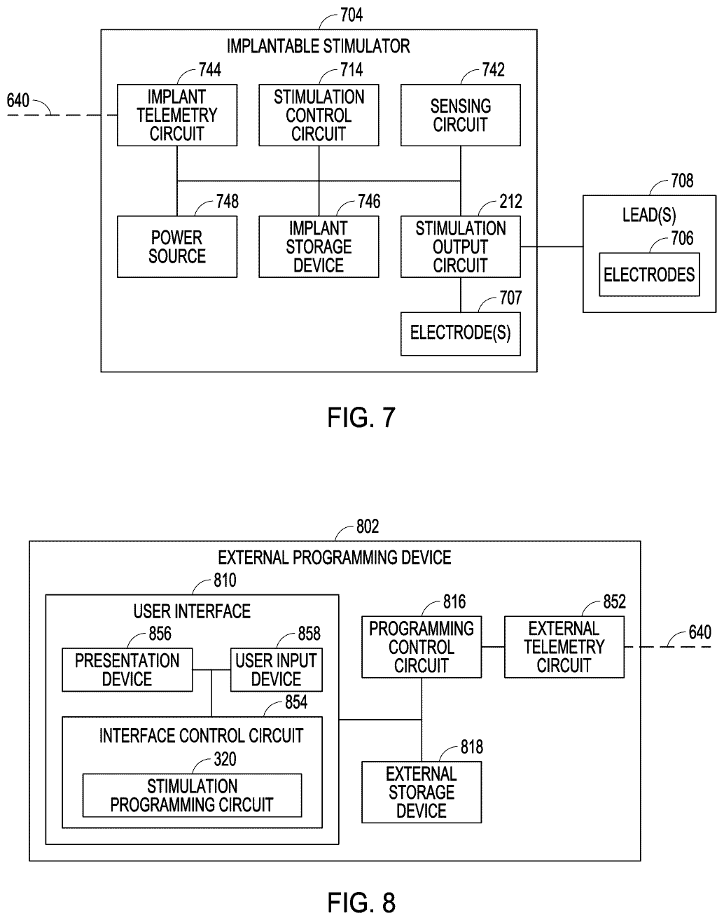

FIG. 7 illustrates an embodiment of implantable stimulator 704 and one or more leads 708 of an implantable neurostimulation system, such as implantable system 600. Implantable stimulator 704 represents an example of stimulation device 104 or 204 and may be implemented, for example, as IPG 604. Lead(s) 708 represents an example of lead system 208 and may be implemented, for example, as implantable leads 608A and 608B. Lead(s) 708 includes electrodes 706, which represents an example of electrodes 106 or 206 and may be implemented as electrodes 606.

Implantable stimulator 704 may include a sensing circuit 742 that provides the stimulator with a sensing capability, stimulation output circuit 212, a stimulation control circuit 714, an implant storage device 746, an implant telemetry circuit 744, a power source 748, and one or more electrodes 707. Sensing circuit 742 can one or more physiological signals for purposes of patient monitoring and/or feedback control of the neurostimulation. In various embodiments, sensing circuit 742 can sense one or more electrospinogram (ESG) signals using electrodes placed over or under the dura of the spinal cord, such as electrodes 706 (which can include epidural and/or intradural electrodes). In addition to one or more ESG signals, examples of the one or more physiological signals include neural and other signals each indicative of a condition of the patient that is treated by the neurostimulation and/or a response of the patient to the delivery of the neurostimulation. Stimulation output circuit 212 is electrically connected to electrodes 706 through one or more leads 708 as well as electrodes 707 and delivers each of the neurostimulation pulses through a set of electrodes selected from electrodes 706 and electrode(s) 707. Stimulation control circuit 714 represents an example of stimulation control circuit 214 and controls the delivery of the neurostimulation pulses using the plurality of stimulation parameters specifying the pattern of neurostimulation pulses. In one embodiment, stimulation control circuit 714 controls the delivery of the neurostimulation pulses using the one or more sensed physiological signals. Implant telemetry circuit 744 provides implantable stimulator 704 with wireless communication with another device such as CP 630 and RC 632, including receiving values of the plurality of stimulation parameters from the other device. Implant storage device 746 can store one or more neurostimulation programs and values of the plurality of stimulation parameters for each of the one or more neurostimulation programs. Power source 748 provides implantable stimulator 704 with energy for its operation. In one embodiment, power source 748 includes a battery. In one embodiment, power source 748 includes a rechargeable battery and a battery charging circuit for charging the rechargeable battery. Implant telemetry circuit 744 may also function as a power receiver that receives power transmitted from an external device through an inductive couple. Electrode(s) 707 allow for delivery of the neurostimulation pulses in the monopolar mode. Examples of electrode(s) 707 include electrode 426 and electrode 418 in IPG 404 as illustrated in FIG. 4.

In one embodiment, implantable stimulator 704 is used as a master database. A patient implanted with implantable stimulator 704 (such as may be implemented as IPG 604) may therefore carry patient information needed for his or her medical care when such information is otherwise unavailable. Implant storage device 746 is configured to store such patient information. For example, the patient may be given a new RC 632 (e.g., by installing a new application in a smart device such as a smartphone) and/or travel to a new clinic where a new CP 630 is used to communicate with the device implanted in him or her. The new RC 632 and/or CP 630 can communicate with implantable stimulator 704 to retrieve the patient information stored in implant storage device 746 through implant telemetry circuit 744 and wireless communication link 640 and allow for any necessary adjustment of the operation of implantable stimulator 704 based on the retrieved patient information. In various embodiments, the patient information to be stored in implant storage device 746 may include, for example, positions of lead(s) 708 and electrodes 706 relative to the patient's anatomy (transformation for fusing computerized tomogram (CT) of post-operative lead placement to magnetic resonance imaging (MRI) of the brain), clinical effect map data, objective measurements using quantitative assessments of symptoms (for example using micro-electrode recording, accelerometers, and/or other sensors), and/or any other information considered important or useful for providing adequate care for the patient. In various embodiments, the patient information to be stored in implant storage device 746 may include data transmitted to implantable stimulator 704 for storage as part of the patient information and data acquired by implantable stimulator 704, such as by using sensing circuit 742.

In various embodiments, sensing circuit 742 (if included), stimulation output circuit 212, stimulation control circuit 714, implant telemetry circuit 744, implant storage device 746, and power source 748 are encapsulated in a hermetically sealed implantable housing or case, and electrode(s) 707 are formed or otherwise incorporated onto the case. In various embodiments, lead(s) 708 are implanted such that electrodes 706 are placed on and/or around one or more targets to which the neurostimulation pulses are to be delivered, while implantable stimulator 704 is subcutaneously implanted and connected to lead(s) 708 at the time of implantation.

FIG. 8 illustrates an embodiment of an external programming device 802 of an implantable neurostimulation system, such as system 600. External programming device 802 represents an example of programming device 102 or 302, and may be implemented, for example, as CP 630 and/or RC 632. External programming device 802 includes an external telemetry circuit 852, an external storage device 818, a programming control circuit 816, and a user interface 810.

External telemetry circuit 852 provides external programming device 802 with wireless communication with another device such as implantable stimulator 704 via wireless communication link 640, including transmitting the plurality of stimulation parameters to implantable stimulator 704 and receiving information including the patient data from implantable stimulator 704. In one embodiment, external telemetry circuit 852 also transmits power to implantable stimulator 704 through an inductive couple.

In various embodiments, wireless communication link 640 can include an inductive telemetry link (near-field telemetry link) and/or a far-field telemetry link (RF telemetry link). This can allow for patient mobility during programming and assessment when needed. For example, wireless communication link 640 can include at least a far-field telemetry link that allows for communications between external programming device 802 and implantable stimulator 704 over a relative long distance, such as up to about 20 meters. External telemetry circuit 852 and implant telemetry circuit 744 each include an antenna and RF circuitry configured to support such wireless telemetry.

External storage device 818 stores one or more stimulation waveforms for delivery during a neurostimulation therapy session, such as a DBS or SCS therapy session, as well as various parameters and building blocks for defining one or more waveforms. The one or more stimulation waveforms may each be associated with one or more stimulation fields and represent a pattern of neurostimulation pulses to be delivered to the one or more stimulation field during the neurostimulation therapy session. In various embodiments, each of the one or more stimulation waveforms can be selected for modification by the user and/or for use in programming a stimulation device such as implantable stimulator 704 to deliver a therapy. In various embodiments, each waveform in the one or more stimulation waveforms is definable on a pulse-by-pulse basis, and external storage device 818 may include a pulse library that stores one or more individually definable pulse waveforms each defining a pulse type of one or more pulse types. External storage device 818 also stores one or more individually definable stimulation fields. Each waveform in the one or more stimulation waveforms is associated with at least one field of the one or more individually definable stimulation fields. Each field of the one or more individually definable stimulation fields is defined by a set of electrodes through which a neurostimulation pulse is delivered. In various embodiments, each field of the one or more individually definable fields is defined by the set of electrodes through which the neurostimulation pulse is delivered and a current distribution of the neurostimulation pulse over the set of electrodes. In one embodiment, the current distribution is defined by assigning a fraction of an overall pulse amplitude to each electrode of the set of electrodes. Such definition of the current distribution may be referred to as “fractionalization” in this document. In another embodiment, the current distribution is defined by assigning an amplitude value to each electrode of the set of electrodes. For example, the set of electrodes may include 2 electrodes used as the anode and an electrode as the cathode for delivering a neurostimulation pulse having a pulse amplitude of 4 mA. The current distribution over the 2 electrodes used as the anode needs to be defined. In one embodiment, a percentage of the pulse amplitude is assigned to each of the 2 electrodes, such as 75% assigned to electrode 1 and 25% to electrode 2. In another embodiment, an amplitude value is assigned to each of the 2 electrodes, such as 3 mA assigned to electrode 1 and 1 mA to electrode 2. Control of the current in terms of percentages allows precise and consistent distribution of the current between electrodes even as the pulse amplitude is adjusted. It is suited for thinking about the problem as steering a stimulation locus, and stimulation changes on multiple contacts simultaneously to move the locus while holding the stimulation amount constant. Control and displaying the total current through each electrode in terms of absolute values (e.g., mA) allows precise dosing of current through each specific electrode. It is suited for changing the current one contact at a time to shape the stimulation like a piece of clay (pushing/pulling one spot at a time).

Programming control circuit 816 represents an example of programming control circuit 316 and generates the plurality of stimulation parameters, which is to be transmitted to implantable stimulator 704, based on a specified neurostimulation program (e.g., the pattern of neurostimulation pulses as represented by one or more stimulation waveforms and one or more stimulation fields, or at least certain aspects of the pattern). The neurostimulation program may be created and/or adjusted by the user using user interface 810 and stored in external storage device 818. In various embodiments, programming control circuit 816 can check values of the plurality of stimulation parameters against safety rules to limit these values within constraints of the safety rules. In one embodiment, the safety rules are heuristic rules.

User interface 810 represents an example of user interface 310 and allows the user to define the pattern of neurostimulation pulses and perform various other monitoring and programming tasks. User interface 810 includes a display screen 856, a user input device 858, and an interface control circuit 854. Display screen 856 may include any type of interactive or non-interactive screens, and user input device 858 may include any type of user input devices that supports the various functions discussed in this document, such as touchscreen, keyboard, keypad, touchpad, trackball, joystick, and mouse. In one embodiment, user interface 810 includes a GUI. The GUI may also allow the user to perform any functions discussed in this document where graphical presentation and/or editing are suitable as may be appreciated by those skilled in the art.

Interface control circuit 854 controls the operation of user interface 810 including responding to various inputs received by user input device 858 and defining the one or more stimulation waveforms. Interface control circuit 854 includes stimulation programming circuit 320.

In various embodiments, external programming device 802 can have operation modes including a composition mode and a real-time programming mode. Under the composition mode (also known as the pulse pattern composition mode), user interface 810 is activated, while programming control circuit 816 is inactivated. Programming control circuit 816 does not dynamically updates values of the plurality of stimulation parameters in response to any change in the one or more stimulation waveforms. Under the real-time programming mode, both user interface 810 and programming control circuit 816 are activated. Programming control circuit 816 dynamically updates values of the plurality of stimulation parameters in response to changes in the set of one or more stimulation waveforms and transmits the plurality of stimulation parameters with the updated values to implantable stimulator 704.

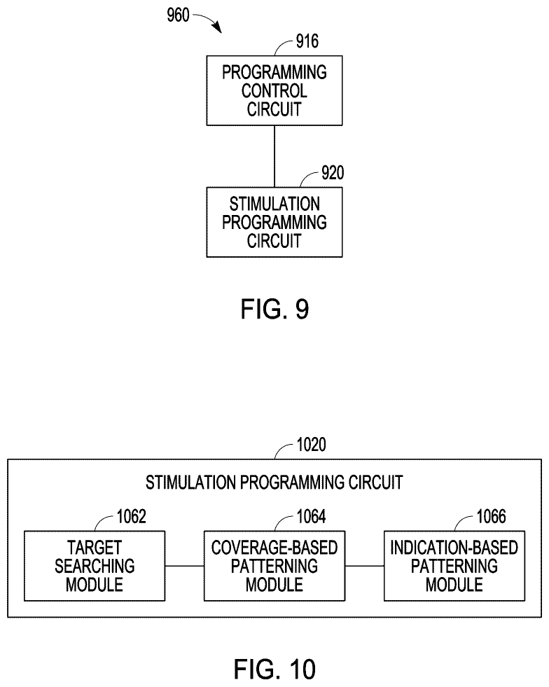

FIG. 9 illustrates an embodiment of a system 960 for programming neurostimulation with one or more dynamic stimulation patterns to be delivered to a targeted area on a patient. System 960 can include a programming control circuit 916 and a stimulation programming circuit 920. System 960 can be implemented in a neurostimulation system such as systems 100, 500, or 600. In various embodiments, system 960 can be implemented in an external system including one or more programming devices, such as programming device 102, programing device 302, external system 502, CP 630 and RC 632, or external programming device 802. For example, when system 960 is implemented in external programming device 802, programming control circuit 916 is implemented in programming control circuit 816, and stimulation programming circuit 920 is implemented in stimulation programming circuit 320. In various embodiments, system 960 can be implemented in a single device or in two or more devices.

Programming control circuit 920 can generate information for programming a stimulation device to control the delivery of the neurostimulation according to a stimulation program specifying stimulation waveform parameters defining at least one stimulation waveform of the neurostimulation and stimulation field parameters defining at least one stimulation field to which the neurostimulation is to be delivered. Examples of the stimulation device include stimulation device 104, stimulation device 204, IPG 404, IPG 504, IPG 604, and implantable stimulator 704. The stimulation program can include one or more stimulation patterns and a schedule specifying timing for each of the one or more stimulation patterns to be applied to control the delivery of the neurostimulation.

Stimulation programming circuit 920 can receive a stimulation field and a stimulation coverage resulting from delivering the neurostimulation to the stimulation field. The stimulation field is defined by the stimulation field parameters. The stimulation coverage is a portion of a targeted area effectively stimulated by the neurostimulation delivered to the stimulation field. In other words, the stimulation coverage is the overlap between the targeted area and the area effectively stimulated by the neurostimulation delivered to the stimulation field. Stimulation programming circuit 920 can determine the stimulation program by including at least one dynamic stimulation pattern configured to expand the stimulation coverage resulting from delivering the neurostimulation to the stimulation field. The dynamic stimulation pattern is defined by at least one time-varying stimulation waveform parameter of the stimulation waveform parameters. In some instances, expansion of the stimulation coverage resulting from delivering the neurostimulation to the stimulation field in this way can achieve efficacy and/or safety levels of a neurostimulation therapy that are not achievable using tonic stimulation patterns.

A “targeted area” can include an area to which neurostimulation is to be delivered for treating a condition of the patient. In SCS, the targeted area can include an area of pain identified on the patient (who may have one or more areas of pain) and/or a location, representation, or other analogue on the spinal cord that is accessible for delivering the neurostimulation to suppress pain. The targeted area can be determined through a target searching process (e.g., a paresthesia-guided target searching process, as discussed below), during which the neurostimulation is delivered to various areas accessible through electrodes placed in or on the patients and connected to a stimulation device. In one embodiment, the targeted area is an area of pain identified on the patient, and system 960 is used to program the stimulation device to deliver an SCS therapy. The stimulation coverage can be represented by a paresthesia coverage, which is a portion of the area of pain where paresthesia is caused by (e.g., perceived by the patient in response to) the neurostimulation delivered to the stimulation field. In other words, the paresthesia coverage is the overlap between the targeted area and the area where paresthesia is caused by the neurostimulation delivered to the stimulation field. It is noted that the intensity of neurostimulation that elicits the paresthesia in the patient during a target searching process may not be chosen as the intensity of neurostimulation for relieving pain during an SCS therapy prescribed the patient. It is also noted that while “area” on the patient, such as in the target area or area of pain, is used herein, aspects of the present subject matter can be applied to a volume (e.g., the area with a depth). For example, pain and paresthesia can be felt by the patient with some depth (e.g., inside the body, limb, back, or the like), and a volume of tissue can be targeted and reached by neurostimulation. Thus, each of such references to an “area” in discussing various aspects of neurostimulation can be applied to a “volume”, in various embodiments.

FIG. 10 illustrates an embodiment of a stimulation programming circuit 1020, which can represent an example of stimulation programming circuit 920. Stimulation programming circuit 1020 can include a target searching module 1062, a coverage-based patterning module 1064, and/or an indication-based patterning module 1066. In the illustrated embodiment, stimulation programming circuit 1020 includes target searching module 1062, coverage-based patterning module 1064, and indication-based patterning module 1066. In various embodiments, stimulation programming circuit 1020 can include any one or any combination of target searching module 1062, coverage-based patterning module 1064, and indication-based patterning module 1066, depending on how the stimulation coverage is determined and the approach used to determine the stimulation program including at least one dynamic stimulation pattern.

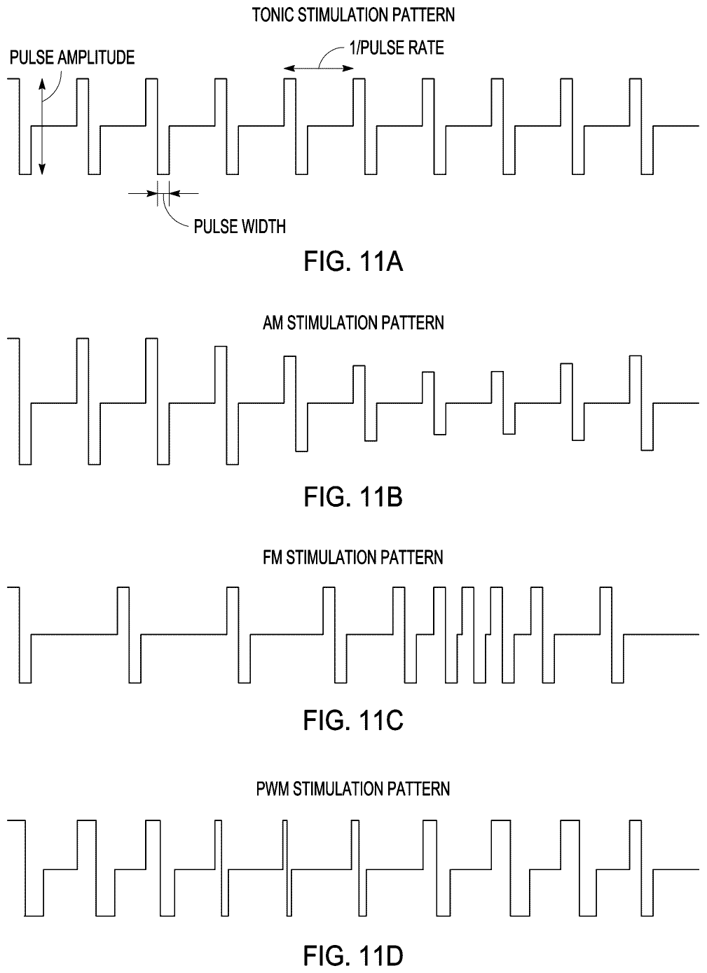

FIGS. 11A-D illustrate examples of various stimulation patterns that can be included in stimulation programs determined using stimulation programming circuit 920 or 1020. FIG. 11A illustrating an example of a tonic stimulation pattern that is a pulse sequence defined using stimulation waveform parameters including a pulse amplitude, a pulse width, and a pulse rate (or pulse frequency, with the pulse period, i.e., 1/pulse rate, shown). The pulse amplitude, the pulse width, and the pulse rate each have constant (not time-varying) values. FIGS. 11B-D each illustrate an example of a dynamic stimulation patter. FIG. 11B illustrates an example of an amplitude-modulated (AM) stimulation pattern in which the pulse amplitude is modulated. FIG. 11C illustrates an example of a frequency-modulated (FM, also referred to as rate-modulated, or RM) stimulation pattern in which the pulse rate is modulated. FIG. 11D illustrates an example of a pulse width-modulated (PWM) stimulation pattern in which the pulse width is modulated.

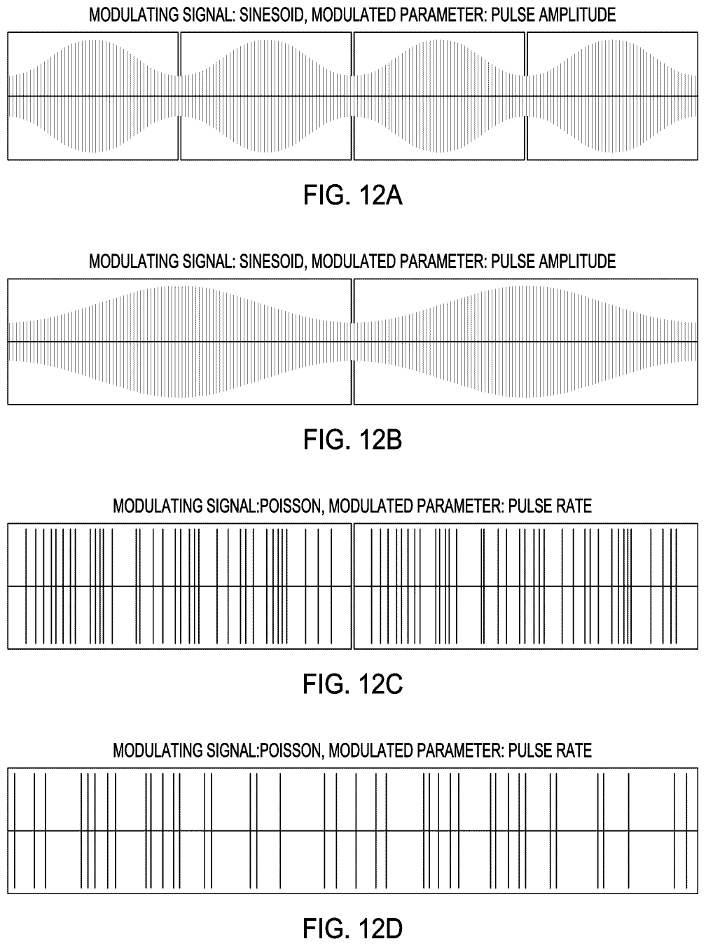

FIGS. 12A-D illustrate examples of various dynamic stimulation patterns each with a modulated stimulation parameter. FIG. 12A illustrates an example of a pulse amplitude modulated by a sinusoidal function. FIG. 12B illustrates another example of a pulse amplitude modulated by a sinusoidal function. FIG. 12C illustrates an example of a pulse rate modulated by a Poisson distribution function. FIG. 12D illustrates another example of a pulse rate modulated by a Poisson distribution function.

Referring back to FIG. 10, target searching module 1062 can determine a stimulation field and a stimulation coverage associated with the stimulation field. The stimulation coverage can be a portion of the targeted area effectively stimulated by the neurostimulation delivered using the stimulation field and a tonic stimulation pattern. The stimulation coverage can be expressed as a percentage of the targeted area. In one embodiment, the targeted area is an area of pain identified to be targeted by an SCS therapy. Target searching module 1062 can determine the stimulation field using paresthesia-guided target searching process in which a paresthesia coverage is determined as the stimulation coverage. The paresthesia coverage is a portion (e.g., expressed as a percentage) of the area of pain where paresthesia is caused by (e.g., perceived by the patient in response to) the neurostimulation delivered using the stimulation field and the tonic stimulation pattern.

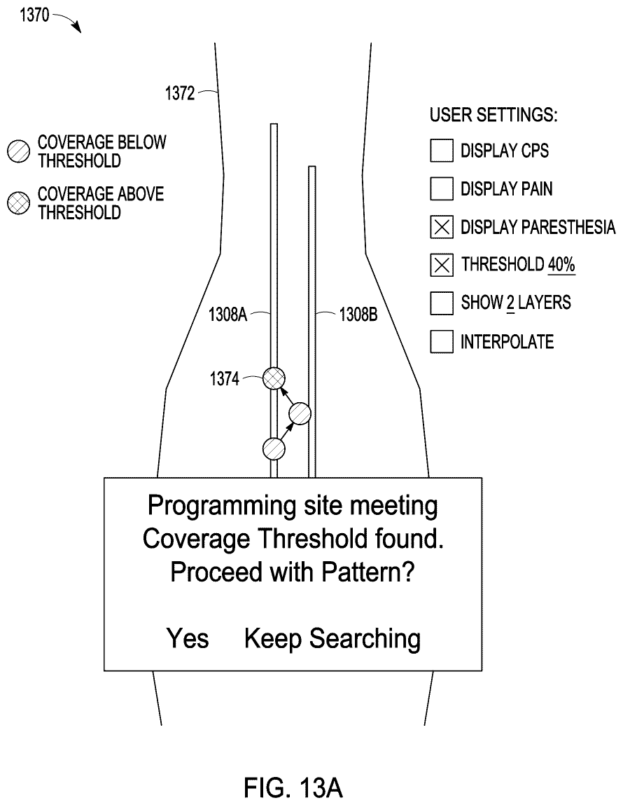

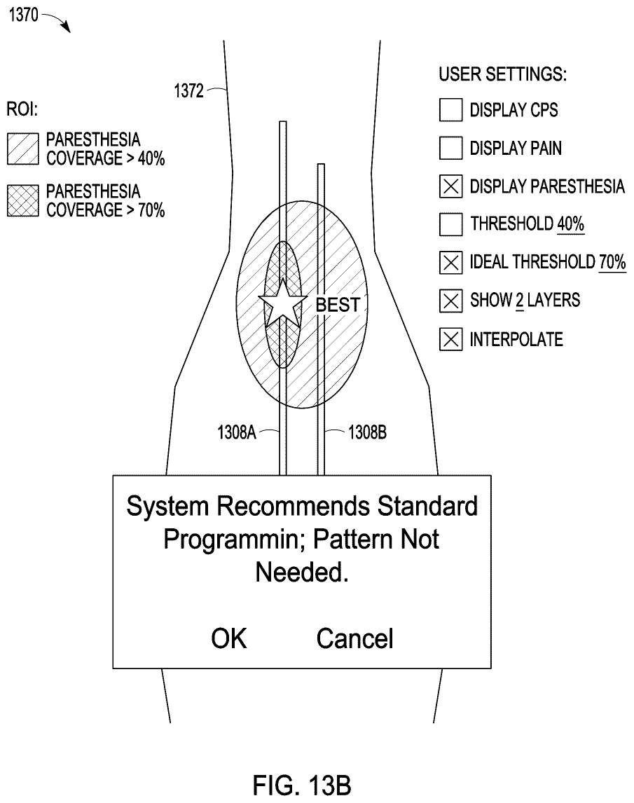

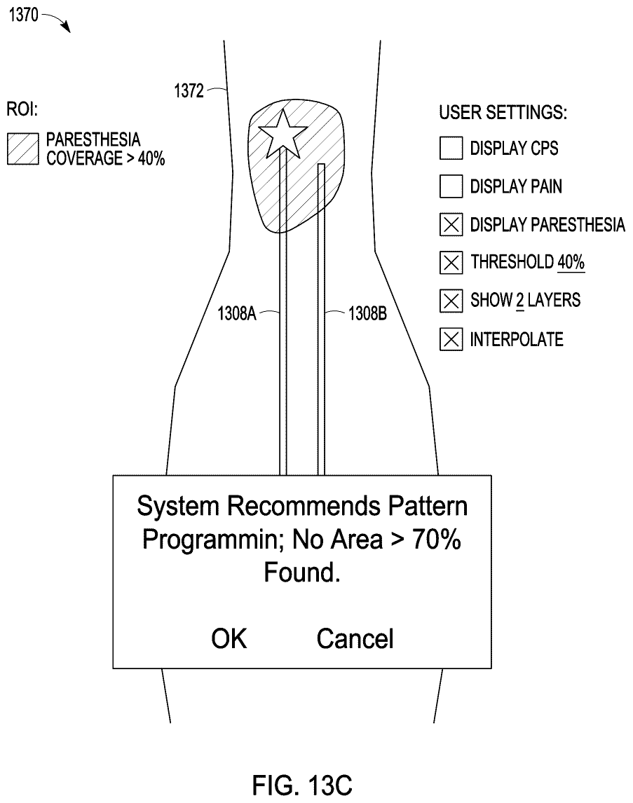

FIGS. 13A-C illustrate an embodiment of a paresthesia-guided target searching process for pain management using spinal cord stimulation (SCS), such as can be performed using target searching module 1062. FIGS. 13A-C each show a paresthesia map 1370 displayed on a screen (e.g., presentation device 856). Paresthesia map 1370 shows a representation of an anatomical region 1372 of the patient including the area of pain (a spinal region as shown) and a representation of electrodes each selectable for delivering the neurostimulation to the patient (two leads 1308A and 1308B as shown, with each lead including stimulation electrodes). Example of the leads 1308A and 1308B include lead system 208, leads 408A-B, lead system 508, lead 608A-B, and lead(s) 708.

FIG. 13A illustrates an example of moving central point of stimulation (CPS) 1374 for an acceptable stimulation coverage. Examples of selectable user settings controlling the display of paresthesia map 1370 as shown in FIG. 13 include: display CPS, display pain, display paresthesia, threshold (paresthesia threshold specified), show (number of) layers (or levels, of paresthesia threshold), and interpolate (otherwise, individual search points, or outline based on search points, is shown). Current and past instances of CPS 1374 (including those associated with paresthesia coverages above and below the paresthesia threshold) can be shown on paresthesia map 1370 to show a history of the target searching. A paresthesia coverage greater than a specified paresthesia threshold (PT, a minimum paresthesia coverage) is obtained after testing one or more sites.

An example of the target searching process performed using target searching module 1062 includes: (1) presenting a representation of an anatomical region (e.g., 1372) of the patient including the area of pain and a representation of electrodes each selectable (e.g., 1308A-B) for delivering the neurostimulation to the patient, (2) receiving a CPS (e.g., 1374) positioned on the anatomical region, (3) determining the stimulation field based on the CPS, (4) causing the stimulation device to deliver the neurostimulation to the stimulation field according to a tonic stimulation pattern, and (5) determining the stimulation coverage (e.g., paresthesia coverage) resulting from the neurostimulation delivered to the stimulation field determined based on the CPS. The performance of the target searching including steps (2)-(5) can be repeated until the stimulation coverage (e.g., the paresthesia coverage) exceeds a specified coverage threshold (e.g., expressed as the percent overlap between the areas of pain and paresthesia). In the example shown in FIG. 13A, the PT is specified to be 40%. When CPS 1374 has moved to a position resulting in a paresthesia coverage greater than 40%, the user is given the option of concluding the target search process and the option of continuing the searching for a higher paresthesia coverage. In various embodiments, the target searching including steps (2)-(5) can be repeated until either the specified coverage threshold (e.g. the PT) is exceeded or an approximately maximum stimulation coverage (e.g., the paresthesia coverage) is reached. The approximately maximum stimulation coverage can be the highest stimulation coverage obtained after testing with a plurality of CPSs.

In various embodiments, with target searching module 1062, the user can enter an “express” programming mode that provides extra-coarse focus and CPS spots. For example, the user can just select best slot or grid out of limited options. In various embodiments, target searching module 1062 can determine fractionalization automatically to determine the stimulation field parameters using a programming algorithm. By using stimulation programs including one or more dynamic stimulation patterns, the present subject matter allow for the target searching process to conclude with a relatively low stimulation coverage (e.g., with the coverage threshold set to about 30%, 40%, or 50%). The relatively low stimulation coverage means a relatively coarse target searching is required, as the user only needs to search until the minimum (as opposed to the best) coverage threshold is exceeded. Upon conclusion of the target searching process, the patient may or may not be asked to provide feedback about pain relief obtained with the best CPS. If the patient is asked to provide the feedback and finds the pain relief provided by using the best CPS and the tonic stimulation pattern to be acceptable or satisfactory, a stimulation program with one or more dynamic stimulation patterns can be considered unnecessary. If the patient finds the pain relief provided by using the best CPS and the tonic stimulation pattern to be unacceptable or unsatisfactory, a stimulation program with one or more dynamic stimulation patterns can be determined to expand the stimulation coverage provided by the best CPS and the tonic stimulation pattern.

FIG. 13B illustrating an example in which a satisfactory stimulation coverage is achieved using the tonic stimulation pattern. The selectable user settings as shown in FIG. 13B further include an ideal threshold and show that the PT is set to 40%, the ideal PT is set to 70%, 2 layers (paresthesia coverage above the PT of 40% and coverage above the PT of 70%) are to be displayed. Regions of interest (RoIs) shown in FIG. 13B include an area of paresthesia that resulted in a paresthesia coverage greater than 40% and an area of paresthesia that resulted in a paresthesia coverage greater than 70%. In one embodiment, as illustrated in FIG. 13B, the paresthesia coverage over 70% is a case in which a tonic stimulation pattern can be sufficient, without a need for one or more dynamic stimulation patterns.

FIG. 13C illustrating an example in which a satisfactory stimulation coverage is not achieved using the tonic stimulation pattern. RoI shown in FIG. 13C includes an area of paresthesia that resulted in a paresthesia coverage greater than 40%, while no area of paresthesia that resulted in a paresthesia coverage greater than 70% is found. A stimulation program with one or more dynamic stimulation patterns can be applied to use one or more time-varying stimulation waveform parameters to compensate for the small paresthesia coverage associated with the stimulation field and constant stimulation waveform parameters.

Referring back to FIG. 10, coverage-based patterning module 1064 can receive a stimulation coverage and to determine a stimulation program using the received stimulation coverage. The stimulation program can include one or more stimulation patterns, which include at least one dynamic stimulation pattern. In one embodiment, coverage-based patterning module 1064 receive the stimulation coverage determined by using target searching module 1062. Coverage-based patterning module 1064 can determine the stimulation program using a coverage-based decision tree mapping values (e.g., percentages) or value ranges of the stimulation coverage to stimulation programs. In one embodiment, coverage-based patterning module 1064 is activated in response to receiving a stimulation coverage that is below a specified satisfactory coverage threshold (e.g., 60%, 70%, or 80%).

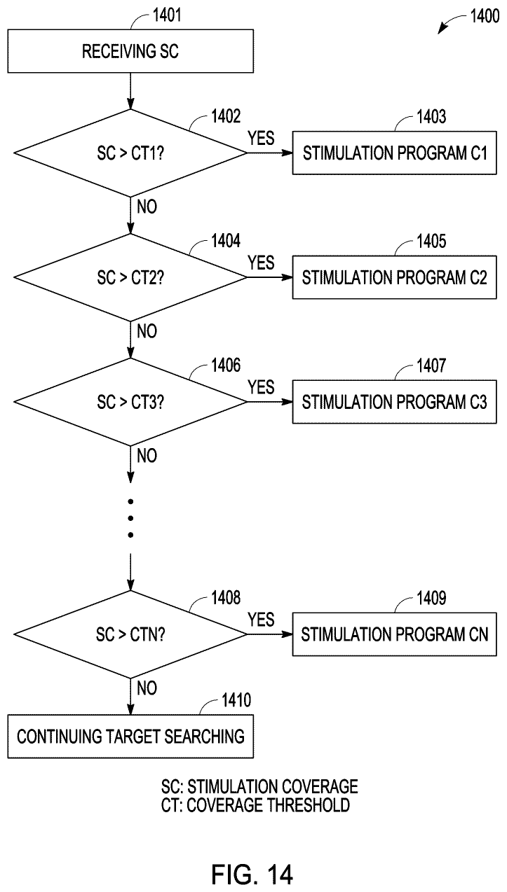

FIG. 14 illustrates an embodiment of a method 1400 for determining a stimulation program including one or more dynamic stimulation patterns based on stimulation coverage of a targeted area achieved by neurostimulation. Method 1400 can be an example of the coverage-based decision tree used by coverage-based patterning module 1064 to determine stimulation programs. In other words, coverage-based patterning module 1064 can be configured to include method 1400 as part of a programming algorithm of stimulation programming circuit 1020. In various examples of existing systems and methods, programming for pain relief relies on maximizing the paresthesia coverage that can be a cumbersome and difficult target searching process, as discussed above. Application of method 1400 can simplify and ease this process through one or multiple approaches, for example: (i) finding an approximate (less precise) stimulation coverage with a single CPS; (ii) starting with a stimulation pattern designed for generating a broad or maximum coverage, and testing and adjusting that pattern until the stimulation coverage covers the area of pain for at least some of the time; and/or (iii) testing a pre-configured stimulation program including various stimulation patterns, mapping the stimulation coverage achieved by each pattern, and confirming that the area of pain is covered by one or more of the various stimulation patterns. Calibration of the stimulation waveform parameters may or may not be necessary with each stimulation pattern and/or stimulation intensity, across patient postures and/or patient activities.

At 1401, a stimulation coverage (SC) is received. The stimulation coverage can result from delivering neurostimulation to a stimulation field according to a tonic stimulation pattern. The stimulation program determined using method 1400 is to be used to control delivery of neurostimulation to the same stimulation field. An example of the stimulation coverage is the paresthesia coverage, with the coverage threshold being the paresthesia threshold (PT).

At 1402, the received SC is compared to a first coverage threshold (CT1). If the received SC exceeds CT1 at 1402, a stimulation program C1 is selected at 1403. If the received SC does not exceed CT1 at 1402, the received SC is compared to a second coverage threshold (CT2) at 1404. If the received SC exceeds CT2 at 1404, a stimulation program C2 is selected at 1405. If the received SC does not exceed CT2 at 1404, the received SC is compared to a third coverage threshold (CT3) at 1406. If the received SC exceeds CT3 at 1406, a stimulation program C3 is selected at 1407. This repeats until the received SC is compared to an Nth coverage threshold (CTN) at 1408. If the received SC exceeds CTN at 1408, a stimulation program CN is selected at 1409. If the received SC does not exceed CTN at 1408, the target searching is continued at 1410 (because the stimulation coverage is below the minimum that can be mapped to a stimulation program. Stimulation programs C1, C2, C3, . . . . CN can be pre-configured stimulation program each including one or more stimulation patterns and each to be mapped to a range of the stimulation coverage. N can be any integer determined based on a desirable resolution of the stimulation coverage. In various embodiments, each of the stimulation programs C1, C2, C3, . . . CN can include one or more stimulation patterns. In one example of such a stimulation program, multiple stimulation patterns are temporally arranged (e.g., cycled through, in a predefined order, or in a user-programmable order), with each stimulation pattern applied for an on-period (during which the neurostimulation is delivered according to the stimulation pattern) and followed by an off-period (during which no neurostimulation is delivered). In various embodiments, the stimulation programs can each be a predetermined “package” or “playlist” of one or more stimulation patterns, or the user can create and/or adjust the contents of each stimulation program using stimulation programming circuit 920 or 1020.

In an example of the coverage-based decision tree mapping ranges of paresthesia coverage to stimulation programs for pain relief, if the paresthesia coverage exceeds 70%, stimulation program C1 is selected (stimulation pattern C1A on for 1 hour, no stimulation for 6 hours, stimulation pattern C1B on for 1 hour, no stimulation for 6 hours, stimulation pattern C1C on for 1 hour, no stimulation for 6 hours, and optionally repeating). Stimulation pattern C1A includes pulses amplitude modulated at 40% depth and delivered at 90 Hz pulse rate, stimulation pattern C1B includes pulses frequency modulated at 40% depth and delivered at 90 Hz pulse rate, and stimulation pattern C1C includes pulses modulated with a first model-inspired modulation function at 20% depth. Modulated at a percentage depth refers to the modulation depth (also known as modulation index) that defines, in percentage, the amount of variation of a carrier signal around resulting from the modulation. If the paresthesia coverage exceeds 60% (but not exceeding 70%), stimulation program C2 is selected (with stimulation pattern C2A on for 2 hours, no stimulation for 24 hours, stimulation pattern C2B on for 2 hours, no stimulation for 24 hours, stimulation pattern C2C on for 2 hours, no stimulation for 24 hours, and optionally repeating).

Stimulation pattern C2A includes pulses amplitude modulated at 40% depth and delivered at 40 Hz pulse rate, stimulation pattern C2B includes pulses modulated with a second model-inspired modulation function at 10% depth, and stimulation pattern C2C includes pulses frequency modulated with a Poisson distribution function and delivered at 90 Hz pulse rate. If the paresthesia coverage exceeds 40% (but not exceeding 60%), stimulation program C3 is selected (with stimulation pattern C3A on for 2 hours, no stimulation for 24 hours, stimulation pattern C3B on for 2 hours, no stimulation for 24 hours, stimulation pattern C3C on for 2 hours, no stimulation for 24 hours, and optionally repeating). Stimulation pattern C3A includes pulses modulated with a combination of modulation functions and delivered at 40 Hz pulse rate, stimulation pattern C3B includes pulses modulated with the second model-inspired modulation function at 10% depth, and stimulation pattern C3C includes non-stimulation. A “combination” of modulation functions in a dynamic stimulation pattern means two or more of the stimulation parameters defining that stimulation pattern are modulated, and different modulation function can be applied to different stimulation parameters. For example, a dynamic stimulation pattern can have a pulse frequency fixed at 40 Hz, a pulse amplitude modulated on a 5-second period at a 50% depth, and a pulse width modulated on a 2-second period at a 20% depth. In another example, in addition to the pulse amplitude and the pulse width, the pulse frequency embodiments, the pulse frequency can also be varied or modulated. In various embodiments, a stimulation program can include a combination of tonic and dynamic stimulation patterns delivered sequentially or concurrently (e.g., through different timing channels of the stimulation device). If the paresthesia coverage does not exceed 40%, the target searching process is to be continued until a better CPS is found to provide a paresthesia coverage that exceeds at least 40%.

Referring back to FIG. 10, indication-based patterning module 1066 can receive an indication of the patient and determine a stimulation program based on the received indication. The indication can be received, for example, using a user input device such as user input device 858. The indication can be selected from a list of indications for each a stimulation program can be determined. In one embodiment, stimulation programs are pre-configured for a list of indications. Indication-based patterning module 1066 presents the list of indications using presentation device 856 and receives the indication of the patient by allowing the user to select from the list of indications using user input device 858. Indication-based patterning module 1066 can determine the stimulation program using the received indication and an indication-based decision tree mapping the list of indications to the pre-configured stimulation programs.

In various embodiments, the pre-configured stimulation programs each include one or more stimulation patterns with at least one dynamic stimulation pattern. The pre-configured stimulation programs can be designated according to various factors of the patient, such as the patient's needs, degree of stimulation coverage, and/or activity level. The patient's pain etiology can be factored into one or more stimulation patterns of the stimulation program. A degree of paresthesia coverage initially selected by the user (if above a PT or marked as “best possible” for the patient) can be factored into selection of one or more stimulation patterns. A survey or decision tree can be used to determine a schedule of stimulation pattern in the stimulation program. Questions can be asked regarding the patient's systemic pain and/or each individual area of pain region. Other symptoms (e.g., those related to autonomic nervous system) can also be included.

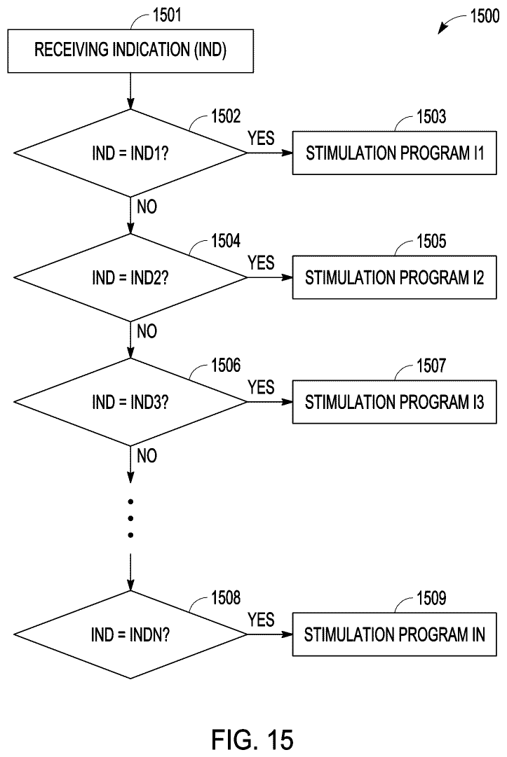

FIG. 15 illustrates an embodiment of a method 1500 for determining a stimulation program including one or more dynamic stimulation patterns based on a patient's indication for neurostimulation. Method 1500 can be an example of the coverage-based decision tree used by indication-based patterning module 1066 to determine stimulation programs. In other words, indication-based patterning module 1066 can be configured to include method 1500 as part of a programming algorithm of stimulation programming circuit 1020. Method 1500 is for customization of stimulation patterns for the patient based on the patient's indications and can be an alternative or a supplemental method of method 1400.

At 1501, an indication of the patient is received. The indication can result from a diagnosis of the patient. At 1502, the received indication (IND) is compared to a first indication (IND1). If the received indication is the first indication at 1502, a stimulation program I1 is selected at 1503. If the IND is not the IND1 at 1402, the IND is compared to a second indication (IND2) at 1504. If the IND is the IND2 at 1504, a stimulation program I2 is selected at 1505. If the IND is not the IND2 at 1504, the IND is compared to a third indication (IND3) at 1506. If the IND is the IND3 at 1506, a stimulation program I3 is selected at 1507. This repeats until the IND is compared to an Nth indication (INDN) at 1508. If the IND is the INDN at 1508, a stimulation program IN is selected at 1509. If the IND is not the INDN at 1508, method 1500 does not determine a stimulation program. Stimulation programs I1, I2, I3, . . . IN can be pre-configured stimulation program each including one or more stimulation patterns and each to be mapped to a range of the stimulation coverage. N is the number of the indications for which stimulation programs are available. In various embodiments, each of the stimulation programs I1, I2, I3, . . . IN can include one or more stimulation patterns. In one example of such a stimulation program, multiple stimulation patterns are cycled through, with each stimulation pattern applied for an on-period (during which the neurostimulation is delivered according to the stimulation pattern) and followed by an off-period (during which no neurostimulation is delivered). In various embodiments, the stimulation programs can each be a predetermined “package” or “playlist” of one or more stimulation patterns, or the user can create and/or adjust the contents of each stimulation program using stimulation programming circuit 920 or 1020.

In an example of the indication-based decision tree mapping indications to stimulation programs for pain relief, if the patient has traditional neuropathic pain, a neuropathic coverage plan is selected, and a paresthesia coverage greater than 60% is sought by performing method 1400. If the patient has diabetic painful neuropathy (DPN), a stimulation program cycling through stimulation patterns I2A, I2B, and I2C is selected, without performing method 1400. Stimulation pattern I2A includes pulses amplitude modulated at 40% depth and delivered at 90 Hz pulse rate for 10 seconds, stimulation pattern I2B includes pulses frequency modulated at 40% depth and delivered at 90 Hz pulse rate for 5 seconds, and stimulation pattern I2C includes pulses pulse width modulated at 30% depth and delivered at 90 Hz pulse rate for 5 seconds). If the patient has nociceptive pain, a nociceptive coverage plan is selected, and a paresthesia coverage greater than 80% is sought by performing method 1400. This repeats until the received indication has been compared to all the indications listed on the coverage-based decision tree.

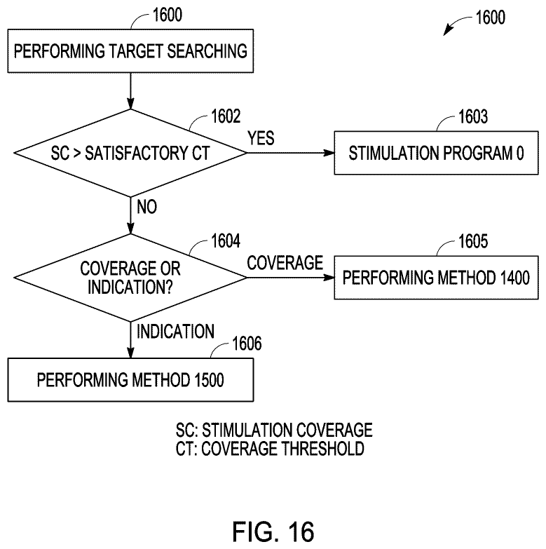

FIG. 16 illustrates an embodiment of a method 1600 for determining a stimulation program using the methods illustrated in FIGS. 13-15. At 1601, a target searching is performed to determine a stimulation field (corresponding to an acceptable or best CPS) and a stimulation coverage resulting from delivering neurostimulation to the stimulation field, for example according to a tonic stimulation pattern. At 1602, the stimulation coverage (SC) is compared to a satisfactory (or “ideal”) coverage threshold (CT). If the SC exceeds the satisfactory CT at 1602, a stimulation program 0 (e.g., including a tonic stimulation pattern) is selected at 1603. If the SC does not exceed the satisfactory CT at 1602, whether to apply the coverage-based decision tree or the indication-based decision tree for determining a stimulation program is selected at 1604. In one embodiment, the user can select at 1604 based on professional judgment on what is more important for the patient. If the coverage-based decision tree is selected at 1604, method 1400 is performed. If the indication-based decision tree is selected at 1604, method 1500 is performed. In one embodiment, method 1500 can be performed to customize the stimulation program for the patient after method 1400 is performed to expand the stimulation coverage.

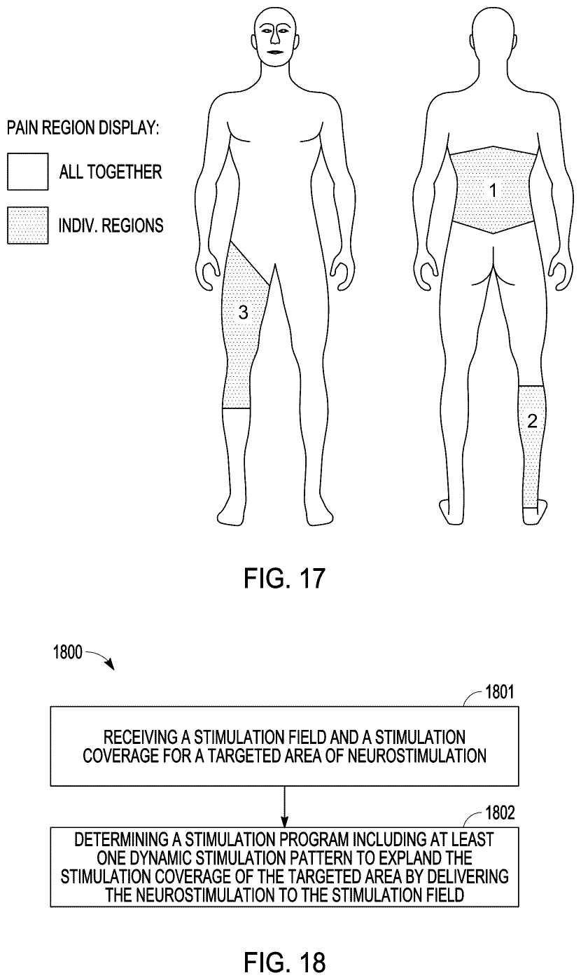

FIG. 17 illustrates an embodiment of a method for presenting a patient's pain regions for determining the stimulation program. As an example, FIG. 17 shows three areas of pain label as pain regions 1, 2, and 3. In various embodiments, the process of determining a stimulation program as discussed with reference to FIGS. 13A-C, 14, 15, and 16 can be performed for each pain region individually or for all the pain regions collectively, depending on the nature of the pain in each pain region and/or capabilities of available stimulation programs.

FIG. 18 illustrates an embodiment of a method 1800 for expanding stimulation coverage achieved by delivering neurostimulation to a stimulation field using one or more dynamic stimulation patterns. In one embodiment, method 1800 can be performed using system 960 as implemented in a neurostimulation system such as system 100, 500, or 600, including various embodiments of their components discussed above. Method 1800 can be performed to determine a stimulation program for programming a stimulation device (e.g., stimulation device 104, stimulation device 204, IPG 404, IPG 504, IPG 604, and implantable stimulator 704), using a programming device (e.g., programming device 102, programming device 302, external system 502, CP 630 and RC 632, or external programming device 802), to control delivery of the neurostimulation to a targeted area on a patient according to the determined stimulation program.

At 1801, a stimulation field and a stimulation coverage for a targeted area of neurostimulation are received. The stimulation coverage resulted from delivering the neurostimulation to the stimulation field. The stimulation coverage is a portion of the targeted area effectively stimulated by the neurostimulation delivered to the stimulation field. In one embodiment, the stimulation field and the stimulation coverage are determined using a target searching process as discussed above with reference to FIGS. 13A-C.

At 1802, a stimulation program is determined. The stimulation program specifies stimulation waveform parameters defining at least one stimulation waveform of the neurostimulation and stimulation field parameters defining the stimulation field. The stimulation program includes at least one dynamic stimulation pattern capable of expanding the stimulation coverage of the target area by delivering the neurostimulation to the stimulation field. The dynamic stimulation pattern is defined by at least one time-varying stimulation waveform parameter of the stimulation waveform parameters. In various embodiments, the stimulation program can be determined by performing methods 1400 and/or method 1500 as discussed above with reference to FIGS. 14 and 15, or method 1600 as discussed above with reference to FIG. 16. In various embodiments, the stimulation program can be determined for each area of pain or for multiple areas of pain, as discussed above with reference to FIG. 17.

In various embodiments, a stimulation program including a predetermined schedule specifying timing of pre-configured stimulation patterns can be assigned to a stimulation field for a given single CPS. A stimulation device can “play through” a sequence of the stimulation patterns according to the schedule. The sequence of stimulation patterns can be configured to improve stimulation coverage (e.g., paresthesia coverage) of the stimulation program and/or improve robustness of the stimulation program to changes (e.g., relative movement between the stimulation electrodes and the targeted tissue).

In various embodiments, stimulation patterns of the stimulation program, including one or more dynamic stimulation pattern, can be adjustable using closed-loop control with a sensed signal as input. Examples of such sensed signal include neural signal including evoked compound action potentials (ECAPs) and accelerometer signal. For example, implantable stimulator 704 can be configured such that sensing circuit 742 can sense the signal as the input, and stimulation control circuit 714 can detect a change in the sensed signal and adjust the one or more stimulation patterns in response to each detection of the change. The adjustment can include adjusting one or more stimulation parameters of a current stimulation pattern and/or transitioning from the current stimulation pattern to another stimulation pattern (e.g., selected based on the type of the change detected). Examples of such change include change of posture of the patient and change of activity level of the patient. The adjustments when performed automatically (e.g., by stimulation control circuit 714) can make the stimulation program robust to the posture and/or activity changes of the patient. In various embodiments, stimulation patterns can be mapped to stimulation coverage, posture of the patient, activity level of the patient, etc., to be selected using the sensed signal (e.g., based on a predefined type of event or change detected from the sensed signal).

In various embodiments, a stimulation program can include dynamic stimulation patterns that cycle in time and/or space. Examples of stimulation parameters that can be modulated for defining dynamic stimulation patterns include: (A) amplitude modulation by sinewave, random signals, or any other function (ramp, exponential, etc.) delivered to one or more electrodes; (B) frequency modulation by sinewave, random signals, or any other function (ramp, exponential, etc.); (C) pulse width modulation by sinewave, random signals, or any other function (ramp, exponential, etc.); (D) any modulation of other parameters such as duty cycle, charge per second, charge per phase, charge density, or others; and (E) any combination of (A)-(D) applied simultaneously. Cycling of stimulation patterns in time can include delivering neurostimulation according to any of the stimulation patterns in (A)-(E) intermittently with on-periods and off-periods that are programmable. The on-periods are time intervals during which the neurostimulation is delivered according to the stimulation patterns. The off-periods are time intervals during which no neurostimulation is delivered. Examples of such on-periods and off-periods include: (a) an on-period of 10 seconds following by an off-period of 1 minute, (b) an on-period of 30 seconds following by an off-period of 10 minutes, (c) an on-period of 10 minutes following by an off-period of 2 hours, (4) an on-period of 1 hour following by an off-period of 22 hours, (5) an on-period of 2 hours following by an off-period of 46 hours, (6) an on-period of 24 hours following by an off-period of 6 days, and (7) an on-period of randomly assigned length following by an off-period of randomly assigned length. Cycling of stimulation patterns in space can include applying a stimulation pattern to a first stimulation filed for a first on-period, then applying the stimulation pattern to a second stimulation filed for a second on-period, then applying the stimulation pattern to a third stimulation filed for a third on-period, etc. The first, second, and third on-period can have the same length or different length and can each be optionally followed by an off-period during which no stimulation is applied to any stimulation field.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering neurostimulation from a stimulation device to a targeted area on a patient, comprising:
a programming control circuit configured to generate information for programming the stimulation device to control the delivery of the neurostimulation according to a stimulation program specifying stimulation waveform parameters defining at least one stimulation waveform of the neurostimulation and stimulation field parameters defining at least one stimulation field to which the neurostimulation is to be delivered; and
a stimulation programming circuit configured to:
receive a stimulation field and a stimulation coverage, the stimulation field defined by the stimulation field parameters, the stimulation coverage being a portion of the targeted area effectively stimulated by the neurostimulation delivered to the stimulation field; and
determine the stimulation program by including at least one dynamic stimulation pattern configured to expand the stimulation coverage resulting from delivering the neurostimulation to the stimulation field, the dynamic stimulation pattern defined by at least one time-varying stimulation waveform parameter of the stimulation waveform parameters.

2. The system of claim 1, wherein the at least one time-varying stimulation parameter is modulated by a modulation function.

3. The system of claim 1, wherein the stimulation programming circuit is configured to determine the stimulation program by including a cycling sequence of different stimulation patterns including the at least one dynamic stimulation pattern.

4. The system of claim 1, wherein the stimulation programming circuit comprises a target searching module configured to determine the stimulation field and to determine a paresthesia coverage as the stimulation coverage, the targeted area is an area of pain, and the paresthesia coverage is a portion of the area of pain where paresthesia is caused by the neurostimulation delivered to the stimulation field.

5. The system of claim 4, comprising a user interface including a presentation device, a user input device, and an interface control circuit coupled to the presentation device and the user input device, the interface device including the stimulation programming circuit, wherein the target searching module is configured to perform a target searching including:
presenting, using the presentation device, a representation of an anatomical region of the patient including the area of pain and a representation of electrodes each selectable for delivering the neurostimulation to the patient;
receiving, using the user input device, a central point of stimulation (CPS) positioned on the anatomical region;
determining the stimulation field based on the CPS;
causing the stimulation device to deliver the neurostimulation to the stimulation field according to a tonic stimulation pattern; and
determining the stimulation coverage resulting from the neurostimulation delivered to the stimulation field determined based on the CPS.

6. The system of claim 5, wherein the target searching module is configured to allow for repeating the performance of the target searching for another CPS until the stimulation coverage exceeds a specified coverage threshold.

7. The system of claim 5, wherein the target searching module is configured to repeat the performance of the target searching for multiple CPSs and determine the stimulation field as the stimulation field resulting in a maximum stimulation coverage identified from the stimulation coverages determined from the repeated performance of the target searching.

8. The system of claim 1, wherein the stimulation programming circuit comprises a coverage-based patterning module configured to receive the stimulation coverage and to determine the stimulation program using the received stimulation coverage and a coverage-based decision tree mapping ranges of the stimulation coverage to pre-configured stimulation programs each including one or more stimulation patterns.

9. The system of claim 8, wherein the coverage-based patterning module is configured to be activated in response to the stimulation coverage being below a specified satisfactory stimulation coverage.

10. The system of claim 1, wherein the stimulation programming circuit comprises an indication-based patterning module configured to receive an indication of the patient selected from a plurality of indications and to determine the stimulation program using the received indication and a coverage-based decision tree mapping the indications to pre-configured stimulation programs each including one or more stimulation patterns.

11. A method for delivering neurostimulation from a stimulation device to a targeted area on a patient, comprising:
receiving a stimulation field and a stimulation coverage, the stimulation coverage being a portion of the targeted area effectively stimulated by the neurostimulation delivered to the stimulation field;
determining, using a processor of a programming device, a stimulation program specifying stimulation waveform parameters defining at least one stimulation waveform of the neurostimulation and stimulation field parameters defining the stimulation field, the stimulation program including at least one dynamic stimulation pattern configured to expand the stimulation coverage resulting from delivering the neurostimulation to the stimulation field, the dynamic stimulation pattern defined by at least one time-varying stimulation waveform parameter of the stimulation waveform parameters; and
programming the stimulation device, using the programming device, to control the delivery of the neurostimulation according to the determined stimulation program.

12. The method of claim 11, further comprising modulating a stimulation parameter using a modulating function to produce the at least one time-varying stimulation parameter.

13. The method of claim 11, further comprising selecting a pre-configured cycling sequence of different stimulation patterns to be the stimulation program, the stimulation patterns each scheduled to be applied for an on-period during which the neurostimulation is to be delivered according to the each stimulation pattern and separated from an adjacent stimulation pattern of the different stimulation patterns by an off-period during which no neurostimulation is delivered.

14. The method of claim 11, further comprising performing a target searching to determine the stimulation field and the stimulation coverage, the target searching including:
presenting an anatomical region of the patient including the targeted area and a representation of electrodes each selectable for delivering the neurostimulation to the patient;
receiving a central point of stimulation (CPS) positioned on the anatomical region;
determining the stimulation field based on the CPS;
causing the stimulation device to deliver the neurostimulation to the stimulation field according to a tonic stimulation pattern; and
determining a stimulation coverage resulting from the neurostimulation delivered to the stimulation field determined based on the CPS.

15. The method of claim 14, wherein the targeted area is an area of pain, and determining the stimulation coverage comprises determining a paresthesia coverage being a portion of the area of pain where paresthesia is caused by the neurostimulation delivered to the stimulation field.

16. The method of claim 14, further comprising repeating the performance of the target searching for another CPS until the stimulation coverage exceeds a specified coverage threshold.

17. The method of claim 11, wherein determining the stimulation program comprises determining the stimulation program using the received stimulation coverage and a coverage-based decision tree mapping ranges of the stimulation coverage to pre-configured stimulation programs each including one or more stimulation patterns.

18. The method of claim 17, further comprising determining the stimulation program using the received stimulation coverage and a coverage-based decision tree in response to the stimulation coverage being below a specified minimum coverage.

19. The method of claim 11, wherein determining the stimulation program comprises:
receiving an indication of the patient selected from a plurality of indications; and
determining the stimulation program using the received indication and an indication-based decision tree mapping the indications to pre-configured stimulation programs each including one or more stimulation patterns.

20. A non-transitory computer-readable storage medium including instructions, which when executed by a system, cause the system to perform a method for delivering neurostimulation from a stimulation device to a targeted area on a patient, the method comprising:
receiving a stimulation field and a stimulation coverage resulting from delivering the neurostimulation to the stimulation field, the stimulation coverage being a portion of the targeted area effectively stimulated by the neurostimulation delivered to the stimulation field;
determining, using a processor of a programming device, a stimulation program specifying stimulation waveform parameters defining at least one stimulation waveform of the neurostimulation and stimulation field parameters defining the stimulation field, the stimulation program including at least one dynamic stimulation pattern configured to expand the stimulation coverage resulting from delivering the neurostimulation to the stimulation field, the dynamic stimulation pattern defined by at least one time-varying stimulation waveform parameter of the stimulation waveform parameters; and
programming the stimulation device, using the programming device, to control the delivery of the neurostimulation according to the determined stimulation program.
